(19) United States
(12) Patent Application Publication
(10) Pub. No.: US 2002/0115202 A1
(43) Pub. Date: Aug. 22, 2002

(54) ADENOVIRAL VECTORS INCLUDING DNA SEQUENCES ENCODING ANGIOGENIC INHIBITORS

(76) Inventors: PAUL HALLENBECK, GAITHERSBURG, MD (US); CHEAUYUN THERESA CHEN, ELLICOTT CITY, MD (US)

Correspondence Address: ELLIOTT M OLSTEIN ESQ, C/O CARELLA BYRNE BAIN GILFILLAN CECCHI, STEWART & OLSTEIN, 6 BECKER FARM ROAD, ROSELAND, NJ, 07068

(21) Appl. No.: 09/373,938

(22) Filed: Aug. 13, 1999

(51) Int. Cl. A61K 48/00; C07H 21/04; C12N 5/06; C12N 15/00

(52) U.S. Cl. 435/320.1; 514/44; 435/328; 536/23.5

(57) ABSTRACT

An adenoviral vector which includes at least one DNA sequence encoding an angiogenic inhibitor, such as endostatin. Such vectors may be employed in treating diseases or disorders associated with angiogenesis, such as cancer, vascular diseases of the eye, including diabetic retinopathy, psoriasis, arthritis, cardiovascular disease, cerebral edema and Kasabach-Merritt syndrome.

This invention relates to adenoviral vectors which may be employed in treating tumors. More particularly, this invention relates to adenoviral vectors including at least one DNA sequence encoding an angiogenic inhibitor and, in particular, endostatin. Such vectors may inhibit, prevent, or destroy the growth of tumors by preventing the formation of blood vessels in such tumors.

### BACKGROUND OF THE INVENTION

Angiogenesis, the process of new blood vessel formation from the existing vessels, is known to be important for tumor growth (Folkman, N. Engl. J. Med., Vol. 285, pgs. 1182-1186 (1971)). Thus, anti-angiogenesis has been studied extensively in cancer therapy applications over the decade. Numerous anti-angiogenesis approaches have been reported and reviewed by Zetter, Ann. Res. Med., Vol. 49, pgs. 407-424 (1998). These include (1) naturally occurring angiogenic inhibitors, e.g., thrombospondin, α-Interferon, platelet factor VI, metalloproteinase inhibitors; (2) synthetic angiogenesis inhibitors, e.g., synthetic protease inhibitors, anti-adhesive peptides (cRGD peptide), anti-integrin antibody (directed against αvβp3); (3) pharmacological inhibitor agents, e.g., TNP470, thalidomide, carboxyamidotrizaole (CAI); and (4) tumor-derived inhibitors, e.g., angiostatin, endostatin. Others are currently either in clinical trial or under development, e.g., anti-signaling agents, and ribozymes inhibiting VEGF receptor synthesis.

Among them, endostatin has been one of the inhibitors demonstrating the most dramatic anti-tumor effect through systemic protein administration (O'Reilly, et al., Cell, Vol. 88, pgs. 277-285 (1997); Boehm, et al., Nature, Vol. 380, pgs. 404-407 (1997)). After several cycles of treatment, the tumors were at their dormancy ultimately resulting in complete tumor arrest. No drug resistance or side effects were reported (O'Reilly, et al., 1997; Boehm, et al., 1997). However, like most angiogenic inhibitors, it functions through cytostatic rather cytotoxic effect, relying on a prolonged maintenance of an anti-angiogenesis state. Direct protein injection may not be sufficient for prolonging maintenance of an anti-angiogenesis state and also likely to be too costly and cumbersome. Thus, the gene therapy approach for anti-angiogenic protein delivery was thought to be more applicable and realistic.

Endostatin is an internal carboxy-terminal peptide of collagen XVIII (O'Reilly, et al., 1997). The crystal structure has been well characterized (Hohenester, et al., EMBO J., Vol. 17, pgs. 1656-1664 (1998); Ding, et al., Proc. Nat. Acad. Sci., Vol. 95, pgs. 10443-10448 (1998)). The protein has been shown to inhibit endothelial cell proliferation in vitro and to have a potent antitumor effect in vivo in several independent studies (O'Reilly, et al. 1997; Dhanabal, et al. Cancer Research, Vol. 57, pgs. 189-197 (1999); Wang, et al., Keystone Symposia, Molecular and Cellular Biology of Gene Therapy A4, 67, 1999). Currently, a Phase I clinical trial with endostatin protein administration is in planning. The molecular mechanism of endostatin induced anti-angiogenesis is not clear. Although the antiangiogenic gene therapy approach has been reported in several studies with various angiogenic inhibitors, e.g., angiostatin (Tanaka, et al., Cancer Research, Vol. 58, pgs. 3362-3369 (1998); Griscelli, et al., Proc. Nat. Acad. Sci., Vol. 95, pgs. 6367-6372 (1998); Nguyen, et al. Cancer Res., Vol. 58, pgs. 56735677 (1998)), platelet factor 4 (Tanaka, et al., Nature Medicine, Vol. 3, pgs. 437-442 (1997)), FLT receptor (Kong, et al., Human Gene Therapy, Vol. 9, pgs. 823-833 (1998)), most of them were designed for in situ delivery. A recent report by Blezinger, et al., Nature Biotechnology, Vol. 17, pgs. 343-348 (Apr. 17, 1999) showed that intramuscular injection of a plasmid including a nucleic acid sequence encoding endostatin could produce circulating endostatin; however, the highest level reached was about 8 ng/ml, which may be insufficient for anti-tumor therapy. Sustained levels of Factor VIII expression using adenoviral vectors has been demonstrated. Systemic delivery of adenoviral vectors encoding an angiogenic inhibitor may provide sustained therapeutic levels of expression to prolong an anti-angiogenesis state.

### SUMMARY OF THE INVENTION

The present invention thus is directed to adenoviral vectors which may be employed for treating tumors by inhibiting the formation of blood vessels in such tumors. Such vectors also may be employed to treat tumor metastases. The adenoviral vectors of the present invention include a DNA sequence encoding endostatin.

### BRIEF DESCRIPTION OF THE DRAWINGS

The invention now will be described with respect to the drawings wherein:

FIG. 1A is a map of plasmid pAvmEndoLxr;

FIG. 1B shows the DNA sequence encoding and the amino acid sequence of murine endostatin, to which is attached a mouse Ig-K leader sequence;

FIG. 2 is a schematic representation of the adenoviral vectors Av3mEndo and Av3NulI;

FIG. 3 shows genomic DNA analysis of Av3mEndo and the control Av3NulI vectors. The isolated DNA was digested with XmnI(X), HindIII(H), ClaI(C) and BamHI(B).

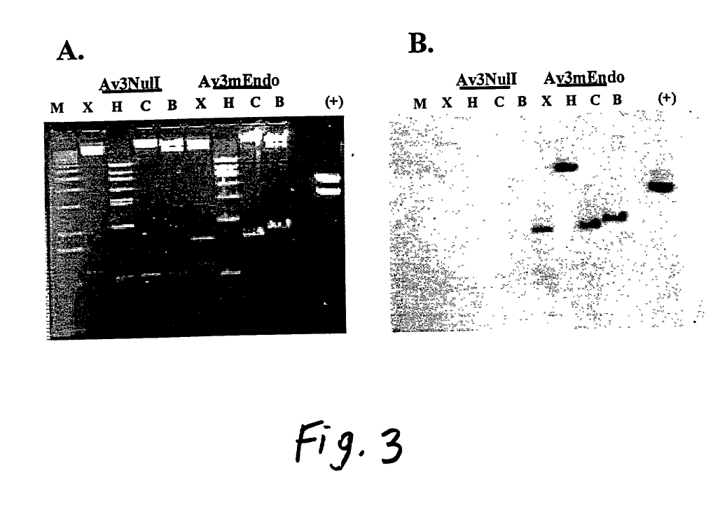

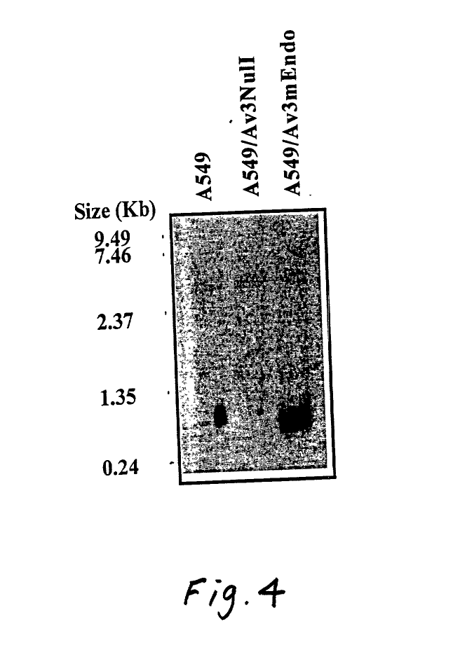

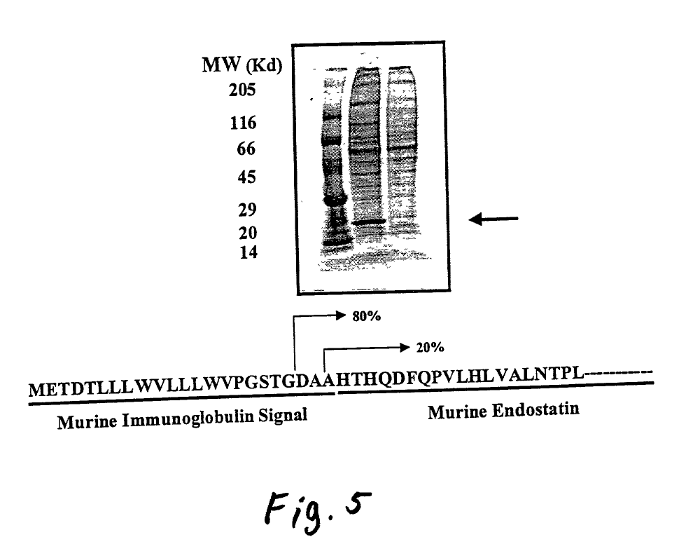

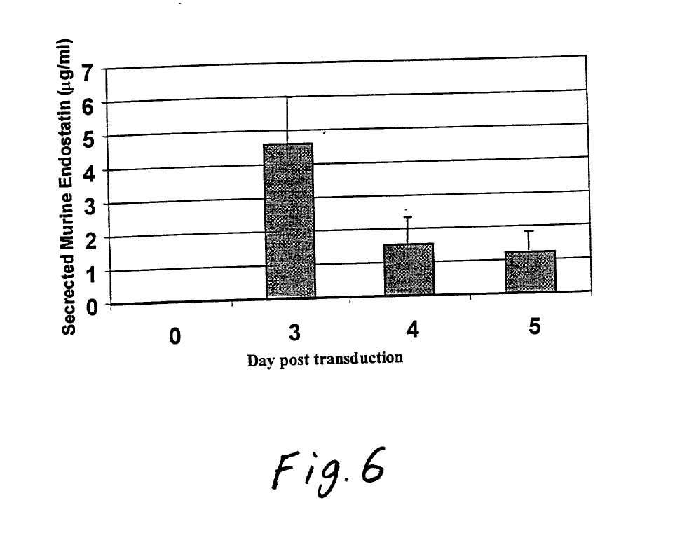

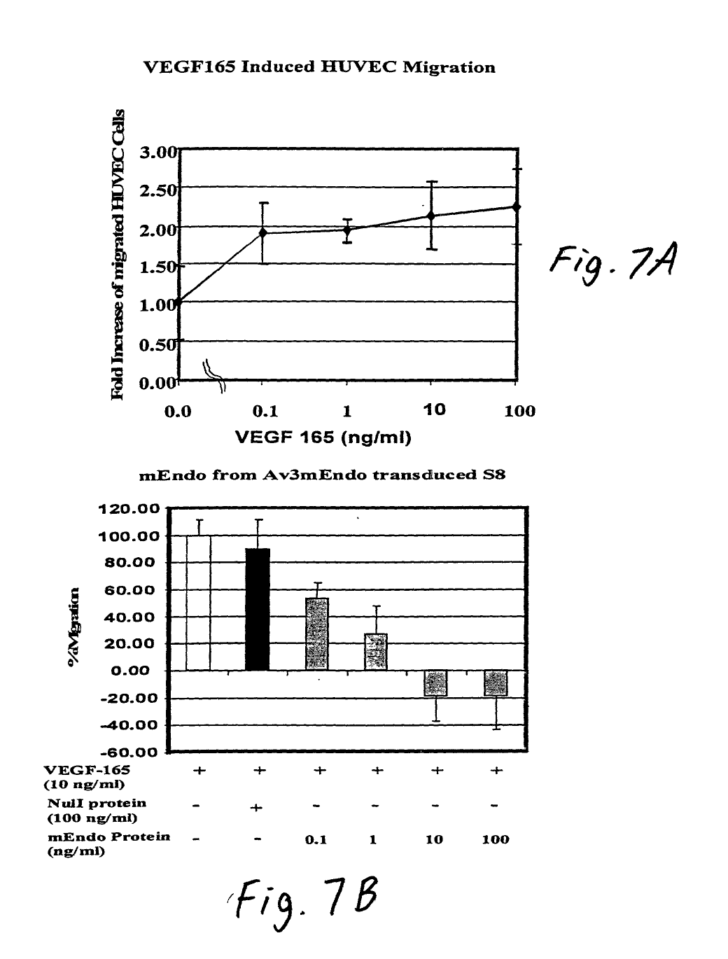

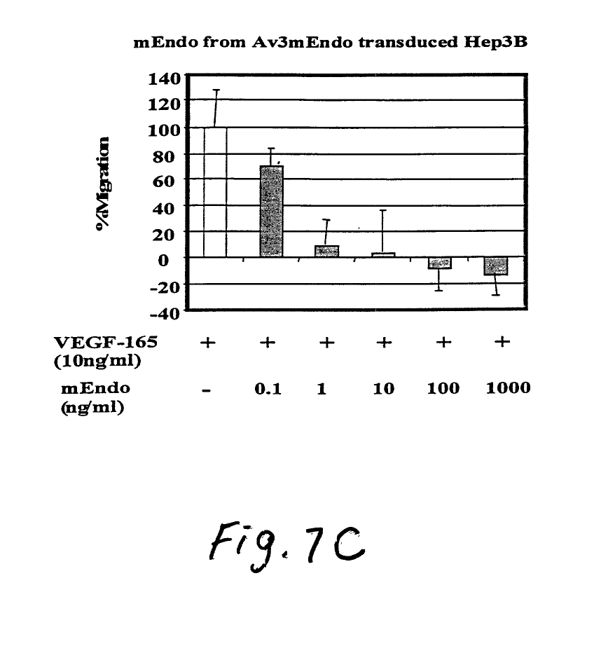

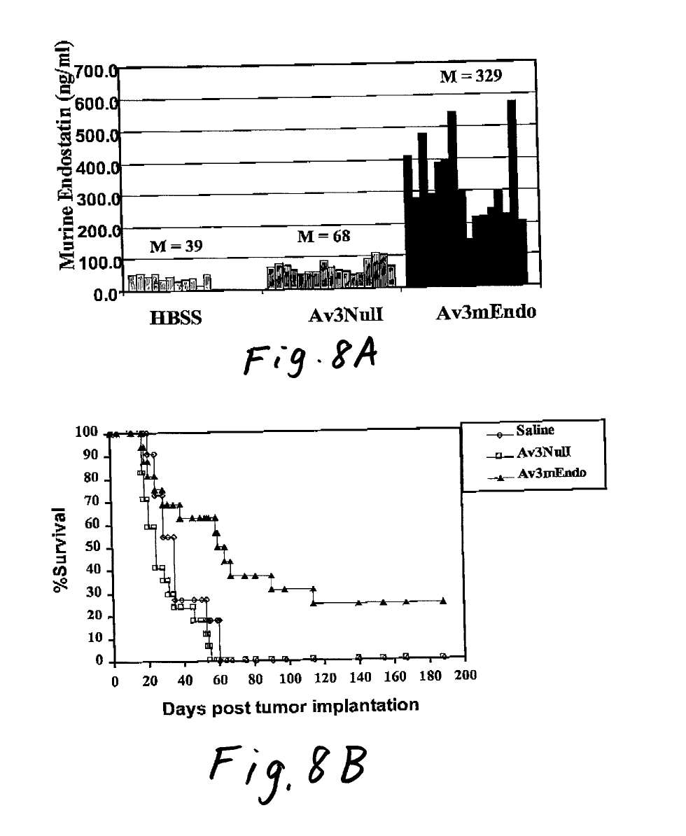

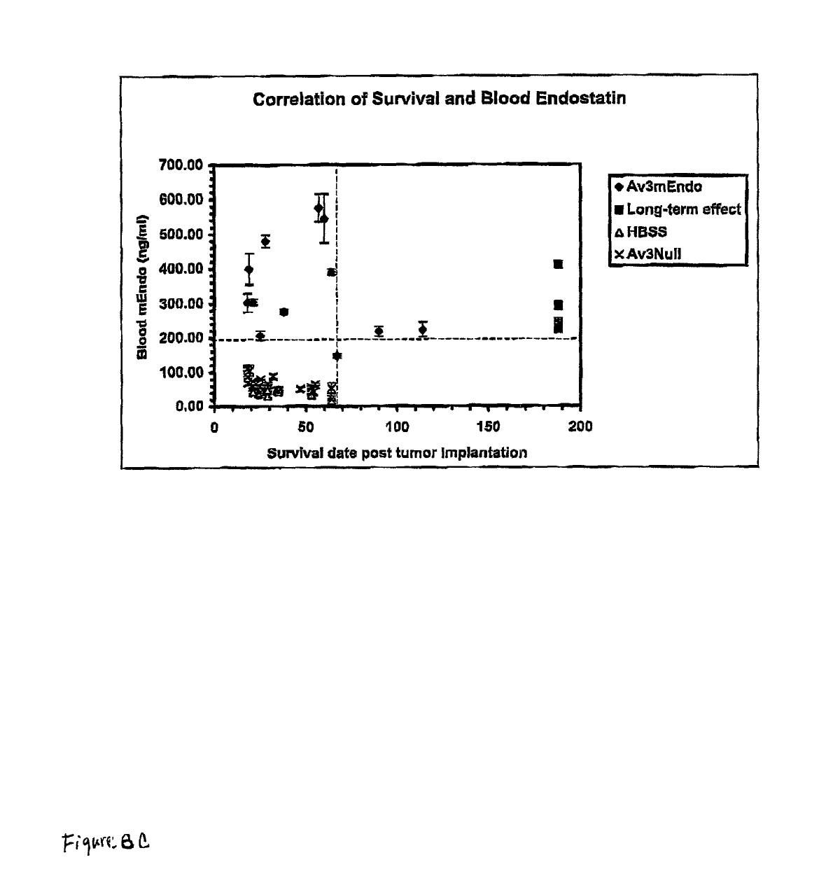

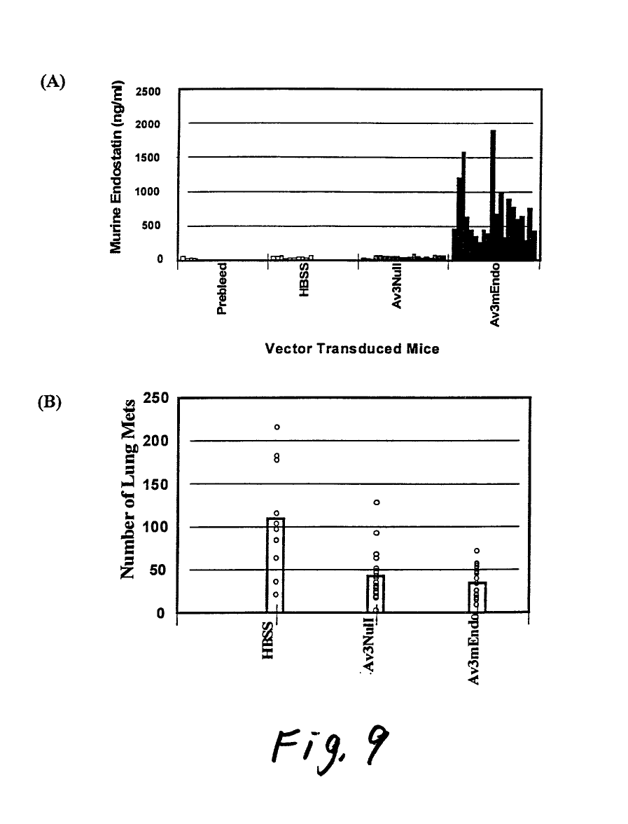

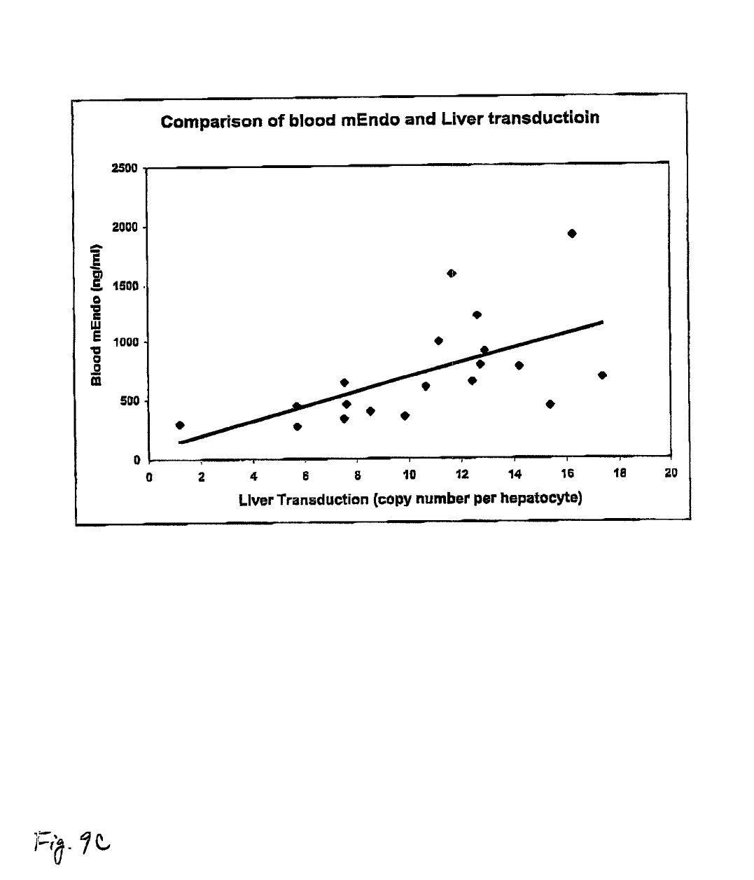

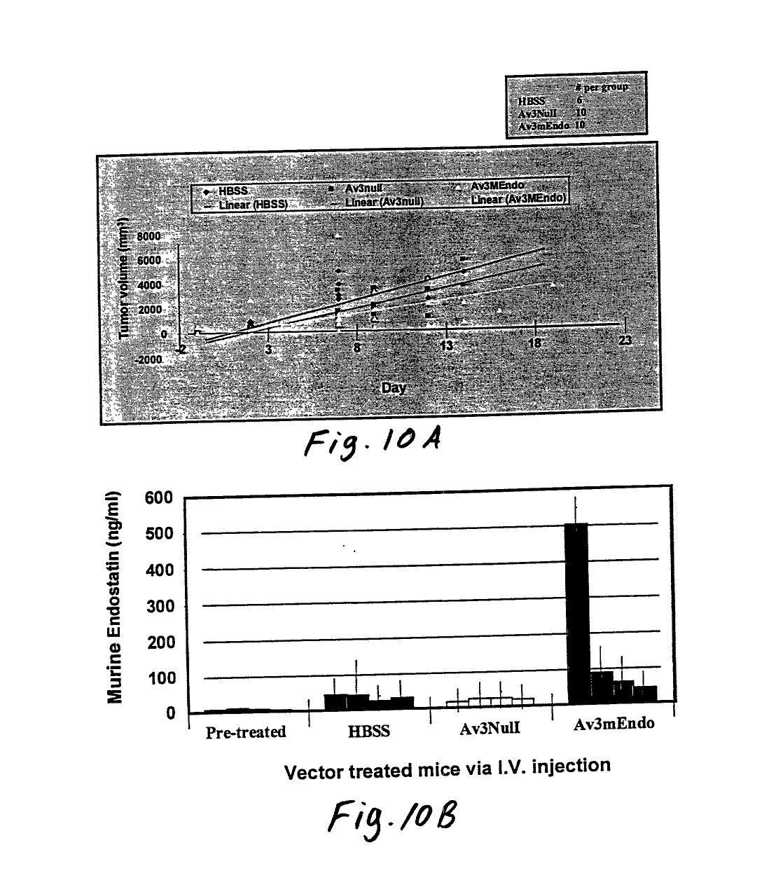

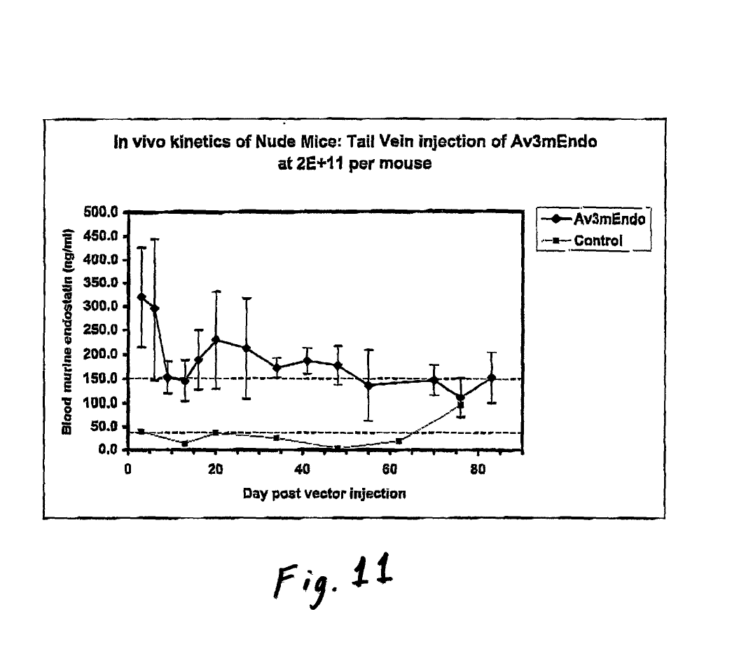

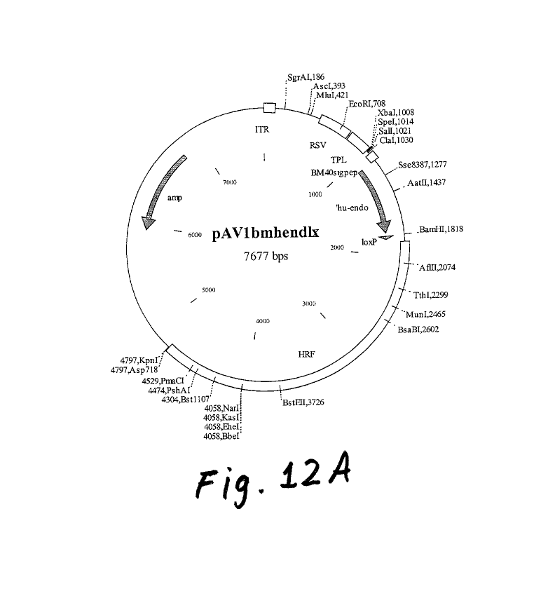

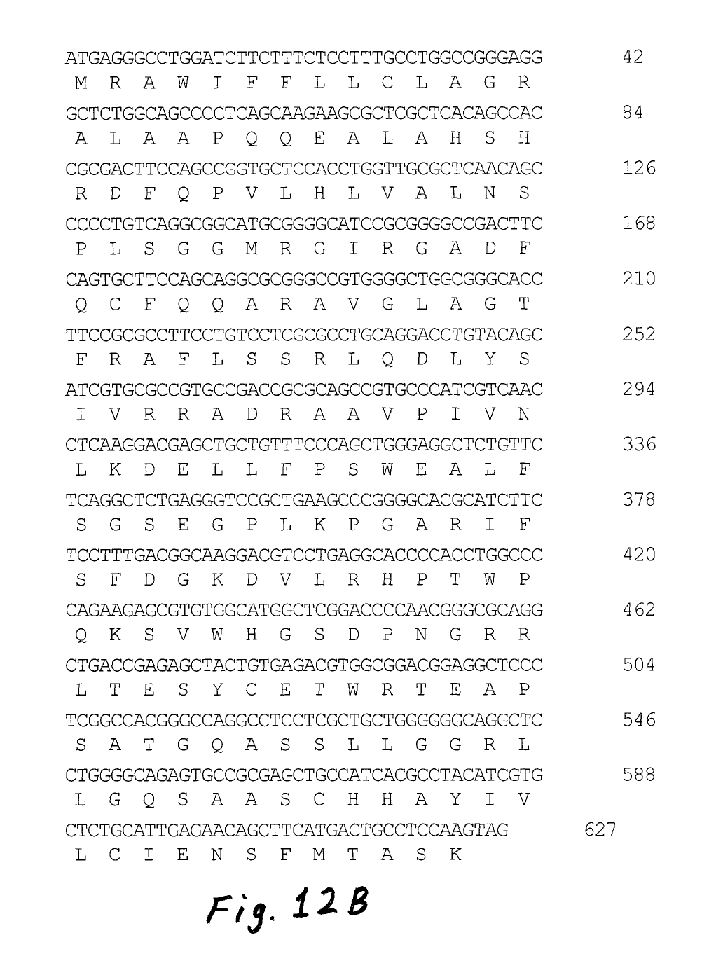

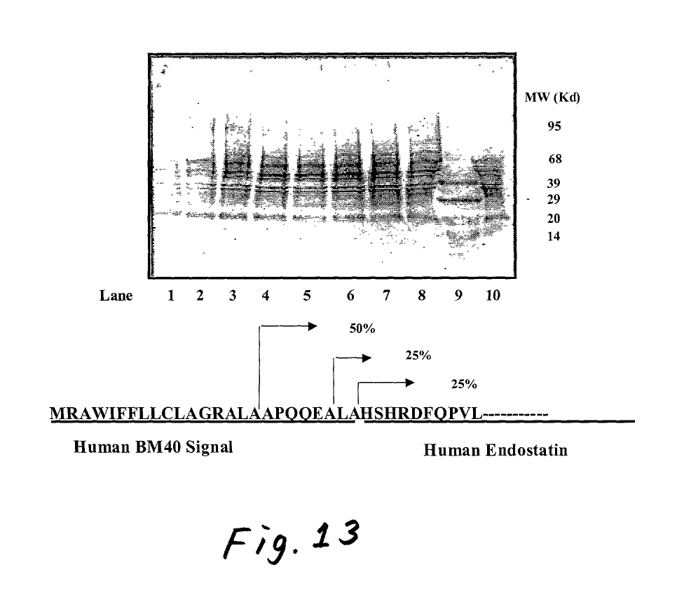

(FIG. 3A) The digested DNA samples (1 μg) were applied to a 1.0% agarose-TAE gel and stained with ethidium bromide to visualize the individual DNA fragments. The 1 kb ladder marker (M) was run in parallel. The pAvmEndoLxr shuttle plasmid encoding murine endostatin was digested with XhoI and used as a positive control (+).

(FIG. 3B) The digested DNA fragments on 1.0% agarose-TAE gel shown in FIG. 3A were transferred to a nylon membrane and hybridized with a ³²P labeled 554 bp internal sig-mEndo probe at approximately 180 μci/ml and exposed to film for 1 hour. Southern Blot analysis with the 554 bp sig-mEndo probe demonstrated the expected hybridization pattern for Av3mEndo;

FIG. 4 is a Northern Blot analysis of adenoviral-mediated expression of murine endostatin in A549 cells.

FIG. 5 shows adenoviral-mediated expression and secretion of murine endostatin in S8 cells. The mEndo (lane 2) and NulI (lane 3) supernatant proteins were analyzed by SDS-PAGE. Each 60 μg supernatant protein was analyzed on 4 to 12% linear gradient pre-casted gel. The protein standard marker was run on lane 1. The gel was stained with Gelcode Blue stain reagent to visualize the protein bands. As indicated, the expected murine endostatin protein band around 20 Kd (marked by arrow) was generated only from Av3mEndo but not from the control Av3NulI. After being transferred to a PVDF membrane from a duplicate SDS-PAGE, the 20 Kd protein band was excised from an immoblin membrane blot and subjected to N-terminal protein sequencing analysis. The determined protein sequence is shown at the bottom with arrows marked as the beginning of the N-termini of two major secreted proteins, 80% containing additional amino acid residues of DAA, and 20% containing residue A from murine Ig-K signal peptide. The results demonstrated that S8 cells transduced with Av3mEndo expressed and secreted murine endostatin after it was processed from murine Ig-K signal peptide;

FIG. 6 is a graph showing secretion of murine endostatin from Av3mEndo transduced Hep3B cells;

FIG. 7 depicts inhibition of migration of HUVEC cells by supernatant protein of Av3mEndo transduced S8 and Hep3B cells. (FIG. 7A) VEGF165 at various concentrations was added to induce HUVEC migration. The migrated HUVEC cells were determined after incubation for 5 hours. The fold of increase of migrated HUVEC cells was calculated relative to the migrated HUVEC cells in the absence of VEGF165.

(FIG. 7B) Supernatant protein was prepared from S8 cells transduced with Av3mEndo or Av3NulI.

(FIG. 7C) Supernatant protein was prepared from Hep3B cells transduced with Av3mEndo or Av3NulI. % migration was calculated based on 100% migration in the presence of 10 μg/ml VEGF165 and in the absence of supernatant protein.

FIG. 8. Colon liver tumor metastasis model. Athymic nude mice were treated with Av3mEndo or control Av3NulI vectors at 2×10¹¹ particles/mouse, or an equal volume of HBSS by tail vein injection.

(FIG. 8A) The individual blood level of endostatin was determined by ELISA analysis. The mean endostatin level (M) in each group was labeled on the top of each group.

(FIG. 8B) The survival was monitored throughout the study based on 100% survival with the number of mice surviving after tumor implantation. Survival times for the mice were correlated to blood endostatin levels (FIG. 8C).

FIG. 9 depicts the results of a B16F10 mouse lung metastasis study. C57BL/6J mice were treated with Av3mEndo (n=20), Av3NulI (n—20), or HBSS (n=12) by tail vein injection at 2×10¹¹ particles per mouse. Two days later, lung metastasis was established by tail vein injection of B16F10 cells at 5×10⁴ cells per mouse. 14 days after tumor implantation, the study was ended.

(FIG. 9A) Blood levels of murine endostatin were determined. Each bar represents the blood level of mEndo from an individual animal.

(FIG. 9B) Surface lung metastasis was determined. Each open circle represents lung metastasis from an individual animal. The bar represents the average lung metastasis of each group with different treatment.

(FIG. 9C). Liver transduction (copy number per hepatocyte) was correlated to blood endostatin levels.

FIG. 10. C57B16/J mice were treated with Av3mEndo (n=10), Av3NulI (n=10), or HBSS (n=6) by tail vein injection at 6×10¹⁰ particles per mouse. Two days later, B16F10 mouse melanoma cells were implanted subcutaneously with 5×10⁵ cells per mouse.

(FIG. 10A) Tumor volume was measured and recorded twice a week. Each symbol represents an individual mouse. The lines represent the average tumor volumes of each group.

(FIG. 10B) Blood level of murine endostatin was determined at the end of the study as described in the text. Each bar represents the blood level of mEndo from an individual animal.

FIG. 11 is a graph showing blood endostatin levels in mice that were given a tail vein injection of 2×10¹¹ particles of Av3mEndo.

FIG. 12A is a map of the plasmid pAV1bmhend1x.

FIG. 12B shows the DNA sequence encoding human endostatin, and the amino acid sequence of human endostatin, to which is attached a human BM40 basement membrane protein leader sequence.

FIG. 13 shows adenoviral-mediated expression and secretion of human endostatin in S8 cells.

### DETAILED DESCRIPTION OF THE INVENTION

In accordance with an aspect of the present invention, there is provided an adenoviral vector including at least one DNA sequence encoding endostatin.

Because endostatin is an internal carboxy-terminal peptide of collagen XVIII, in a preferred embodiment, the adenoviral vector further includes a DNA sequence encoding a secretion signal peptide immediately 5′ to the DNA sequence encoding endostatin. Such secretion signal peptides include, but are not limited to, the secretion signal peptide of Ig-kappa, the secretion signal peptides of α-factors, and the secretion signal peptide of the human basement membrane protein BM-40.

The DNA sequence encoding endostatin is under the control of a suitable promoter. It is to be understood, however, that the scope of the present invention is not to be limited to any specific promoters.

The adenoviral vector which is employed may, in one embodiment, be an adenoviral vector which includes essentially the complete adenoviral genome (Shenk et al., Curr. Top. Microbiol. Immunol., 111(3): 1-39 (1984). Alternatively, the adenoviral vector may be a modified adenoviral vector in which at least a portion of the adenoviral genome has been deleted.

In a preferred embodiment, the adenoviral vector comprises an adenoviral 5′ ITR; an adenoviral 3′ ITR; an adenoviral encapsidation signal; a DNA sequence encoding endostatin, and a promoter controlling the DNA sequence encoding endostatin. The vector is free of at least the majority of adenoviral E1 and E3 DNA sequences, but is not free of all of the E2 and E4 DNA sequences, and DNA sequences encoding adenoviral proteins promoted by the adenoviral major late promoter.

In one embodiment, the vector also is free of at least a portion of at least one DNA sequence selected from the group consisting of the E2 and E4 DNA sequences.

In another embodiment, the vector is free of at least the majority of the adenoviral E1 and E3 DNA sequences, and is free of a portion of the other of the E2 and E4 DNA sequences.

In still another embodiment, the gene in the E2a region that encodes the 72 kilodalton binding protein is mutated to produce a temperature sensitive protein that is active at 32° C., the temperature at which the viral particles are produced. This temperature sensitive mutant is described in Ensinger, et al., J. Virology, 10:328-339 (1972), Van der Vliet et al., J. Virology, 15:348-354 (1975), and Friefeld, et al., Virology, 124:380-389 (1983).

Such a vector, in a preferred embodiment, is constructed first by constructing, according to standard techniques, a shuttle plasmid which contains, beginning at the 5′ end, the “critical left end elements,” which include an adenoviral 5′ ITR, an adenoviral encapsidation signal, and an E1a enhancer sequence; a promoter (which may be an adenoviral promoter or a foreign promoter); a multiple cloning site (which may be as herein described); a poly A signal; and a DNA segment which corresponds to a segment of the adenoviral genome. The promoter may, in one embodiment, be a regulatable promoter, such as, for example, a glucororticoid-responsive promoter or an estrogen-responsive promoter, or the promoter may be a tissue—specific promoter. The vector also may, in another embodiment, contain genomic elements which may increase and/or maintain expression of the DNA sequence encoding endostatin. Such genomic elements include, but are not limited to, introns, exons, polyadenylation sequences, and 5′ and 3′ untranslated regions. Such genomic elements, and representative examples thereof, also are described in U.S. Pat. No. 5,935,935, issued Aug. 10, 1999. The vector also may contain a tripartite leader sequence. The DNA segment which corresponds to a segment of the adenoviral genome serves as a substrate for homologous recombination with a modified or mutated adenovirus, and such sequence may encompass, for example, a segment of the Adenovirus 5 genome no longer than from base 3329 to base 6246 of the genome. The plasmid may also include a selectable marker and an origin of replication. The origin of replication may be a bacterial origin of replication. Representative examples of such shuttle plasmids include pAvS6, which is described in published PCT Application Nos. W094/23582, published Oct. 27, 1994, and W095/09654, published Apr. 13, 1995 and in U.S. Pat. No. 5,543,328, issued Aug. 6, 1996. The DNA sequence encoding endostatin then may be inserted into the multiple cloning site to produce a plasmid vector.

This construct is then used to produce an adenoviral vector. Homologous recombination is effected with a modified or mutated adenovirus in which at least the majority of the E1 and E3 adenoviral DNA sequences have been deleted. Such homologous recombination may be effected through co-transfection of the plasmid vector and the modified adenovirus into a helper cell line, such as 293 cells, by CaPO₄ precipitation. Upon such homologous recombination, a recombinant adenoviral vector is formed that includes DNA sequences derived from the shuttle plasmid between the NotI site and the homologous recombination fragment, and the DNA derived from the E1 and E3 deleted adenovirus between the homologous recombination fragment and the 3′ ITR.

In one embodiment, the homologous recombination fragment overlaps with nucleotides 3329 to 6246 of the Adenovirus 5 (ATCC VR-5) genome.

Through such homologous recombination, a vector is formed which includes an adenoviral 5′ ITR, an adenoviral encapsidation signal; an E1a enhancer sequence; a promoter; a DNA sequence encoding endostatin; a poly A signal; adenoviral DNA free of at least the majority of the E1 and E3 adenoviral DNA sequences; and an adenoviral 3′ ITR. The vector also may include a tripartite leader sequence. The vector may then be transfected into a helper cell line, such as the 293 helper cell line (ATCC No. CRL1573), which will include the E1a and the E1b DNA sequences, which are necessary for viral replication, and to generate adenoviral particles. Transfection may take place by electroporation, calcium phosphate precipitation, microinjection, or through proteoliposomes.

In another embodiment, the adenoviral vector is free of all or a portion of each of the adenoviral E1 and E4 DNA sequences, or is free of all or a portion of each of the adenoviral E1 and E2 DNA sequences, or is free of all or a portion of each of the E1, E2, and E4 DNA sequences.

Such vectors may be assembled by direct in vitro ligation from combinations of plasmids containing portions of modified or unmodified virus genome or plasmids and fragments derived directly from a linear adenoviral genome, such as the Adenovirus 5 genome (ATCC No. VR-5) or Adenovirus 5 derived viruses containing mutations or deletions.

In another alternative, the vectors can be assembled by homologous recombination, within a eukaryotic cell, between a plasmid clone containing a portion of the adenoviral genome (such as the Adenovirus 5 genome or the adenovirus 5 E3-mutant Ad d1327 (Thimmapaya, et al., Cell, Vol. 31, pg, 543 (1983)) with the desired modifications, and a second plasmid (such as, for example pAvS6), containing the left adenoviral ITR, an E1 region deletion, and the desired transgene. Alternatively, homologous recombination may be carried out between a plasmid clone and a fragment derived directly from a linear adenovirus (such as Adenovirus 5, or Ad d1327 or an Adenovirus 5 derived virus containing mutations or deletions) genome.

The vector then is transfected into a cell line capable of complementing the function of any essential genes deleted from the viral vector, in order to generate infectious viral particles. The cell line in general is a cell line which is injectable and able to support adenovirus or adenoviral vector growth, provide for continued virus production in the presence of glucocorticoid hormones, and is responsive to glucocorticoid hormones (i.e., the cell line is capable of expressing a glucocorticoid hormone receptor). Cell lines which may be transfected with the essential adenoviral genes, and thus may be employed for generating the infectious adenoviral particles include, but are not limited to, the A549, KB, and Hep-2 cell lines.

Because the expression of some viral genes may be toxic to cells, the E1 region, as well as the E2b, and/or E4 regions, may be under the control of an inducible promoter. Such inducible promoters may include, but are not limited to, the mouse mammary tumor virus (MMTV) promoter (Archer, et al., Science, Vol. 255, pgs. 1573-1576 (Mar. 20, 1992)); the synthetic minimal glucocorticoid response element promoter GRE5 (Mader, et al., Proc. Nat. Acad. Sci., Vol. 90, pgs. 5603-5607 (June 1993)); or the tetracycline-responsive promoters (Gossen, et al., Proc. Nat. Acad. Sci., Vol. 89, pgs. 5547-5551 (June 1992)). In another alternative, the E1 region is under the control of inducible promoter, and the E2a, E2b and/or E4 regions are under the control of their native promoters. In such alternative, the native promoters are transactivated by expression of the E1 region.

In one embodiment, the cell line includes the entire adenoviral E4 region with its native promoter region, and the E1 a region or the entire E1 region (including the E1 a and E1b regions) under the control of a regulatable or inducible promoter, such as, for example, the mouse mammary tumor virus (or MMTV) promoter, which is a hormone inducible promoter, or other such promoters containing glucocorticoid responsive elements (GRE's) for transcriptional control. In another embodiment, the E4 DNA sequence also is expressed from a regulatable promoter, such as the MMTV promoter. The E1 and E4 DNA sequences may be included in one expression vehicle, or may be included in separate expression vehicles. Preferably, the expression vehicles are plasmid vectors which integrate with the genome of the cell line.

Such vectors, wherein the vector is free of all or a portion of each of the adenoviral E1 and E4 DNA sequences, or is free of all or a portion of each of the adenoviral E1 and E2 DNA sequences, or is free of all or a portion of the E1, E2 and E4 DNA sequences, and the complementing cell lines, also are described in PCT Application No. WO96/18418, published Jun. 20, 1996, the contents of which are incorporated herein by reference.

The adenoviral vector of the present invention may be administered to a host in vivo in an amount effective to inhibit angiogenesis in a host. The host may be a mammalian host, including human and non-human primate hosts.

The adenoviral vector may be administered to a mammalian host in an amount effective to provide endostatin levels of up to 1,000,000 ng/ml, or 1 mg/ml. Although the adenoviral vector may be administered to a mammalian host in an amount effective to provide endostatin levels of up to 1,000,000 ng/ml. Applicants have found that endostatin, when expressed by mammalian cells transduced with the adenoviral vector of the present invention, significantly is more active (about 1,000 times more active) than endostatin expressed by non-mammalian cells, such as yeast cells or bacterial cells such as E. coli cells. Thus, in order to achieve a desired anti-angiogenic effect, one can provide a mammalian host with endostatin at lower levels by administering the adenoviral vector to a mammalian host, as opposed to providing a mammal with significantly greater levels of endostatin expressed by yeast or bacteria.

In one embodiment, when administered to a mammalian host, the adenoviral vector is administered in an amount effective to provide endostatin levels which are from about 2 to 5 times the basal levels of endostatin. In general, in such an embodiment, the adenoviral vector is administered to a mammalian host in an amount effective to provide for expression of endostatin at a level of at least about 200 ng/ml, preferably from about 200 ng/ml to about 500 ng/ml.

In another embodiment, the adenoviral vector is administered in an amount of from about 10⁸ plaque forming units to about 10¹⁴ plaque forming units, preferably from about 10⁸ plaque forming units to about 10¹¹ plaque forming units, more preferably from about 10⁹ plaque forming units to about 10¹⁰ plaque forming units.

In general, the adenoviral vectors are administered systemically, such as for example, by intravenous administration (such as, for example, portal vein injection or peripheral vein injection), or intraarterial administration, including hepatic artery administration. The adenoviral vectors also may be administered intraperitoneally.

The adenoviral vectors may be administered in combination with a pharmaceutically acceptable carrier suitable for administration to a patient. The carrier may be a liquid carrier (for example, a saline solution), or a solid carrier, such as, for example, microcarrier beads.

Cells which may be infected by the adenoviral vector include, but are not limited to, endothelial cells, and in particular, endothelial cells of blood vessels, and more particularly, endothelial cells of blood vessels of tumors, hepatocytes, and lung cells.

In one embodiment, the adenoviral vectors may be used to infect liver cells, or hepatocytes, whereby the liver cells will express endostatin. The liver can serve as a secretory organ for the systemic delivery of therapeutic proteins. Also, the liver parenchyma is accessible readily to large molecules, such as DNA sequences or recombinant viruses present in the blood. Thus, the liver is an appropriate organ for the delivery of the adenoviral vectors of the present invention. The endostatin expressed by such hepatocytes inhibits, prevents, or destroys the growth of endothelial cells of blood vessels, and in particular the endothelial cells of blood vessels of tumors, thereby preventing angiogenesis in the tumors. By preventing angiogenesis in the tumor, growth of the tumor is inhibited, prevented, or destroyed.

Tumors which may be treated with the adenoviral vectors of the present invention include, but are not limited to, solid tumors of the colon, lung, prostate, breast, stomach, intestine, rectum, mouth, brain, cervix, liver, esophagus, bladder, ovary, uterus and head and neck cancer. The adenoviral vectors also may be employed to treat lymphoma and leukemia.

The adenoviral vector also may be employed in treating tumor metastases, including metastases of the tumors mentioned hereinabove, and in particular to treating tumors which have metastasized to the liver. The vector may be administered in amounts such as those hereinabove described.

The liver is a prime target organ for cancer metastases which include mainly bronchogenic, prostate, gastrointestinal, and certain gynecological tumors. When the adenoviral vectors infect liver cells, the liver metastatic tumors may be the first tumors the secreted endostatin encounters at high regional concentrations, after liver cells are infected by the adenoviral vectors. Thus, by infecting liver cells with the adenoviral vectors, one can provide for the delivery of endostatin to a prime target organ for cancer metastases.

The adenoviral vectors are applicable particularly to the treatment of colon cancer metastases, and in particular to colon cancer which has metastasized to the liver. For example, adenoviral vectors including a DNA sequence encoding endostatin are administered systemically to a patient. The adenoviral vectors may be administered in amounts such as those hereinabove described. The adenoviral vectors infect liver cells, whereby the liver cells secrete endostatin. The endostatin secreted by the liver cells inhibits, prevents, or destroys the growth of endothelial cells of the blood vessels of the metastasized tumor. By inhibiting, preventing, or destroying the growth of endothelial cells of the tumor, the endostatin stops the blood supply to tumor cells, thereby inhibiting, preventing, or destroying the growth of the metastasized tumor.

In an other embodiment, the adenoviral vectors may be employed in treating tumors or tumor metastases, by administering the adenoviral vectors regionally. The adenoviral vectors may be administered in amounts such as those hereinabove described. The terms “regionally” and “regional administration,” as used herein, mean that the adenoviral vector is administered to an area proximal to the tumor or tumor metastasis, or is administered to the organ containing the tumor or tumor metastasis. For example, the adenoviral vectors may be employed in treating prostate cancer by administering the adenoviral vectors directly to the prostate. The adenoviral vectors will infect prostate cells, whereby the infected prostate cells secrete endostatin. The endostatin secreted by the prostate cells inhibits, prevents, or destroys the growth of endothelial cells of the blood vessels of the prostate cancer. By inhibiting, preventing, or destroying the growth of endothelial cells of the prostate cancer, the endostatin stops the blood supply to the prostate cancer cells, thereby inhibiting, preventing, or destroying the growth of the prostate cancer.

In addition, tumor metastases may be treated by administering the adenoviral vector to an organ containing the tumor metastasis. For example, in treating a tumor metastasis in the liver, one would administer the adenoviral vectors to the liver, whereby the adenoviral vectors will infect liver cells, and whereby the infected liver cells secrete endostatin. Alternatively, the adenoviral vectors may be administered to the portal vein or hepatic artery, whereby the adenoviral vectors will travel to and infect liver cells. The endostatin secreted by the liver cells inhibits, prevents, or destroys the growth of endothelial cells of the blood vessels of the tumor metastasis. Thus, the endostatin stops the blood supply to the tumor metastasis cells, thereby inhibiting, preventing, or destroying the growth of the tumor metastasis.

The adenoviral vectors of the present invention also may be employed to treat other diseases and disorders associated with angiogenesis. Such diseases and disorders include, but are not limited to, neovascular diseases of the eye, including diabetic retinopathy, cardiovascular disease, arthritis, psoriasis, cerebral edema, and intravascular coagulopathy (Kasabach-Merritt syndrome).

Such diseases or disorders may be treated by administering the adenoviral vectors systemically, as hereinabove described, or by administering the adenoviral vectors regionally; i.e., to an area proximal to the disease or disorder, or to the organ affected by the disease or disorder.

In another embodiment, the adenoviral vectors which include at least one DNA sequence encoding endostatin may be administered to an animal in order to use such animal as a model for studying a disease or disorder and the treatment thereof. For example, an adenoviral vector containing a DNA sequence encoding an angiogenic inhibitor may be given to an animal having a disease or disorder associated with angiogenesis. Subsequent to the administration of such vector containing the at least one DNA sequence encoding endostatin, the animal is evaluated for expression of endostatin. From the results of such a study, one then may determine how such adenoviral vectors may be administered to human patients for the treatment of the disease or disorder associated with angiogenesis.

In yet another embodiment, the adenoviral vectors including at least one DNA sequence encoding endostatin may be employed to transduce cells in vitro, whereby the transduced cells produce endostatin in vitro.

In a preferred embodiment, the adenoviral vectors may be employed to transduce mammalian cells. Applicants have found that, as stated hereinabove, when the adenoviral vectors of the present invention transduce mammalian cells, the endostatin expressed by such mammalian cells significantly is more active than endostatin expressed by non-mammalian cells, such as by yeast cells or bacterial cells such as E. coli cells, for example. In particular, endostatin expressed by mammalian cells, transduced with the adenoviral vector in vitro, has an effective concentration which inhibits migration of endothelial cells in vitro by 50% (EC50) of 0.17 ng/ml±0.06 ng/ml, as compared with an EC50 of about 2,500 ng/ml for endostatin expressed by yeast cells or bacterial cells.

Mammalian cells which may be transduced with the adenoviral vectors include, but are not limited to, A549 cells and Hep3B cells.

### EXAMPLES

The invention now will be described with respect to the examples; however, the scope of the present invention is not intended to be limited thereby.

### Example 1

Materials and Methods

Cell Lines

Human umbilical vein endothelial cells (HUVEC) were obtained from Cascade Biologics, Inc. (Portland, Oreg.) and were cultured in M200 supplemented with Growth Supplement for Large Vessel Endothelial Cells (LSGS) (Cascade Biologics, Inc). A549 (ATCC No. CCL-185) (human lung carcinoma), Hep3B (human hepatocellular carcinoma) (ATCC No. HB-8064), 293 (human embryonic kidney) cells (ATCC No. CRL-1573) were cultured routinely in Richter's CM with 5% FBS; Eagle's Minimal Essential Medium (EMEM) with 10% FBS; and Richter's CM with 10% FBS respectively. S8 (derived from A549 cells and described in PCT Application No. WO97/25446, published Jul. 17, 1997) were made at Genetic Therapy, Inc., Gaithersburg, Md. and cultured in Richter's CM with 5% FBS.

PCR and Assembly of Murine endostatin cDNA and Ig-kappa Leader Sequence

The mouse endostatin cDNA was PCR amplified from mouse collagen XVIII clone ID #748987 from GenomeSystems, Inc. with the primers of 5′-ACT GGT GAC GCG GCC CAT ACT CAT CAG GAC TTT CAG CC-3′ and 5′-AAG GGC TAT CGA TCT AGC TGG CAG AGG CCT AT-3′ (598 bp F1 fragment). The mouse Ig-kappa leader was PCR amplified from pSecTag2 (Invitrogen) with the primers of 5′-CAC TGC TTA CTG GCT TAT CG-3′ and 5′-CTG ATG AGT ATG GGC CGC ACC AGT GG-3′ (147 bp F2 fragment). PCR was carried out with Pfu DNA polymerase (Stratagene) for 35 cycles in the following conditions: 95° C. hot start for 3 min., 95° C. denaturation for 1 min, 55° C. annealing for 1 min, and 72° C. extension for 2 min. The DNA fragments were gel purified.

The sig-mEndo chimeric DNA (718 bp) was generated by PCR splice overlap extension (Horton, et al., Biotechniques, Vol. 8, pgs. 528-535 (1990)) with F1 and F2 DNA fragments generated above as templates to assemble mouse Ig-kappa leader sequence and murine endostatin cDNA. PCR was carried out with the primers of 5′-ACT GGT GAC GCG GCC CAT ACT CAT CAG GAC TTT CAG CC-3′ and 5′-CTG ATG AGT ATG GGC CGC GTC ACC AGT GG-3′ using Pfu DNA polymerse (Stratagene, LaJolla, Calif.). PCR was run for 35 cycles in the following conditions: 95° C. hot start for 3 min., 95° C. denaturation for 1 min, 60° C. annealing for 1 min, and 72° C. extension for 2 min.

Plasmid Av1H9FR (described in PCT application No. W094/29471, published Dec. 22, 1994) was digested with SfiI, followed by T4 polymerase overhang modification and religation, to form AvS17H9F. Plasmid AvS17H9F was cut with BamHI, and a loxP site was inserted into the BamHI site to create a pAvF91xr. The 718 bp sig-mEndo chimeric DNA fragment was gel purified and cloned into the Nhel and ClaI sites of the adenoviral shuttle plasmid, pAvF91xr, to create pAvmEndoLxr. The sig-mEndo chimeric DNA was cloned downstream of the RSV promoter and the adenoviral tripartite leader and included the SV40 polyadenylation signal, a homologous recombination region, and LoxP site for Cre/Lox mediated recombination. The entire region of the sig-mEndo in the pAvmEndoLxr plasmid was confirmed with the direct sequencing analysis by Gene Therapy Core Technologies Molecular Core Laboratory.

Construction of Recombinant Adenoviral Vectors, Av3mEndo

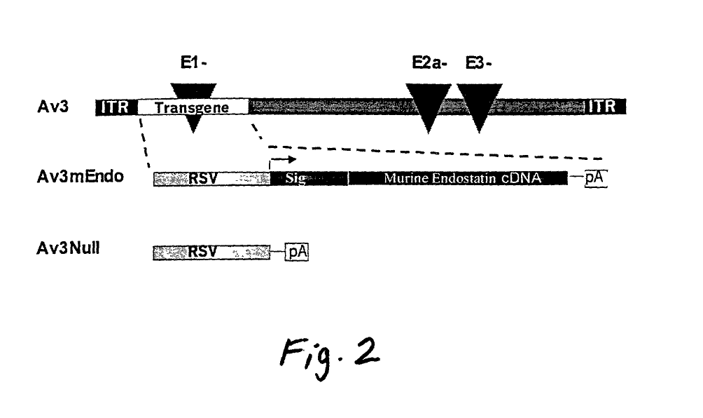

The recombinant Av3mEndo (with E1, E2a, and E3-deletion, a schematic of which is shown in FIG. 2) encoding the sig-mEndo chimeric protein was generated by the “Quick Cre/Lox two plasmid system” in the following procedure. The plasmids pAvmEndoLxr and pSQ3 were linearized first with NotI and ClaI restriction enzymes, respectively. The plasmid pSQ3 includes, in a 5′ to 3′ direction, a loxP site and a portion of the Adenovirus 3 genome which includes the hexon and fiber coding sequences and a 3′ ITR. The transient transfection was performed on the 6-well plate at 4×10⁵ of 293 cells per well using the calcium phosphate mammalian transfection system (Promega Corporation, Madison, Wis.). The calcium phosphate-DNA precipitate was prepared with 4.8 pg of linearized pAvmEndoLxr, 12 μg of linearized pSQ3, 6 μg of pcmvCre, and 6 μg of pcmvE2a in a total volume of 1.8 ml. The plasmid pcmvCre includes a CMV promoter, a T7 promoter priming site, the Cre recombinase open reading frame, a bovine growth hormone polyadenylation signal, a zeocin resistance open reading frame, and an ampicillin resistance gene. The plasmid is constructed by inserting a KpnI-XbaI fragment including the Cre open reading frame into pcDNA3.1Zeo+ (Invitrogen), which was cut with KpnI and XbaI. Plasmid pcmvE2a includes a CMV promoter, a T7 promoter priming site, the Adenovirus 5 E2a open reading frame, a bovine growth hormone polyadenylation signal, a bacteriophage F1 origin of replication, an SV40 promoter and origin of replication, a neomycin resistance gene, an SV40 polyadenylation signal, an E coli origin of replication, and an ampicillin resistance gene. The plasmid was constructed by inserting the PCR-Amplified E2a coding region (1,603 bp), including 7bp upstream of the ATG to the termination codon, TAA, with the addition of a BamHI site (5′ end) and an XbaI site (3′ end) inserted into pcDNA3.1 (Invitrogen) digested with BamHI and XbaI. 0.6 ml of calcium phosphate-DNA precipitate was added to each well. The 293 cells were incubated with calcium phosphate-DNA precipitate at 37° C. for 16 hours. The precipitate was removed and the cells were washed with PBS. Fifteen days post transfection, the cytopathic effect (CPE) was observed. The cells and the medium were then harvested by scraping. The crude viron lysate was prepared by five cycles of freezing and thawing. The Av3mEndo vector was re-amplified in S8 cells with 0.3 μM dexamethasone in Richcter's CM medium containing 5% FBS until CPE was observed.

Av3mEndo Research Seed Lot Characterization

The research seedlot purified Av3mEndo vector was scaled up by the Genetic Therapy, Inc., Gene Therapy Core Technologies. The adenoviral vector titer (particles/ml) and biological titer (pfu/ml) were determined as described (Mittereder, et al., J. Virol., Vol. 70, pgs. 7498-7509 (1996)) by Genetic Therapy, Inc., Gene Therapy Core Technologies. The ratio of total particles to infectious particles (particles/pfu) was calculated. The purity of research seedlot preparation, defined as an assessment of possible contamination with replication competent adenovirus (RCA) was assessed by Genetic Therapy, Inc., Gene Therapy Core Technologies. The Av3mEndo seedlot was determined to be negative for RCA.

Characterization of Recombinant Av3mEndo

Various restriction digest by XmnI, HindII, ClaI, and BamHI of the purified viral DNA of Av3mEndo, and Av3NulI were compared on 1.0% agarose-TAE gel to verify the genome structure and purity. Southern Blot analysis from the duplicate run was carried out following the standard protocol. After transfer to a nylon membrane, the membrane was prehybridized in 0.5 M NaPO₄, 1 mM EDTA, 0.5% BSA, 7% SDS at 65° C. for 2 hours. The sig-mEndo internal probe for both Southern and Northern analyses was prepared from pAvmEndoLxr digested by XbaI and StuI at 37° C. for 4 hours and gel purified. The membrane was then hybridized with a 554 bp [³²P]-labeled sig-mEndo internal probe at 65° C. following the standard protocol. Membrane was exposed to film for one hour at room temperature.

Northern Blot Analysis

A549 cells were transduced with Av3mEndo, or control Av3NulI vector at the pfu to cell ratio of 1. Forty-eight hours post transduction, the cell pellets were harvested and total RNA was isolated using the RNAzol B (Tel-Test, Inc., Friendswood, Tex.) extraction method. Northern Blot analysis was carried out according the standard procedure. A 20 μg aliquot of total RNA was analyzed on 1% Agarose formaldehyde/MOPS gel. RNA was transferred to a nylon membrane and prehybridized in Zip Hyb Solution at 60° C. for 45 min. The membrane was then hybridized with a 554 bp [³²P]-labeled sig-mEndo internal probe at 65° C. and washed in SSC/SDS containing buffers at 65° C. following the standard protocol. Membrane was exposed to X-ray film for 18 hours at −70° C.

Preparation of mEndo and NulI Supernatant Proteins from Vector Transduced Cells

The mEndo and NulI supernatant proteins were prepared from Av3mEndo and Av3NulI transduced S8 or Hep3B cells, respectively. Seventy-two hours post transduction, the supernatant was collected and filtered through 2μ filter. Each 40 ml of supernatant was passed through 1-ml heparin Sepharose CL-6B (Amersham Pharmacia Biotech, Piscataway, N.J.) equilibrated with 50 mM Tris-C1, pH 7.5, 0.1 M NaCl, and 10% glycerol. After unbound protein washed with 50 mM Tris-C 1, pH 7.5, 0.1 M NaCl, and 10% glycerol, the heparin column bound protein was eluted with 4-ml buffer containing 50 mM Tris-Cl, pH 7.5, 1 M NaCl, and 20% glycerol. The protein concentration was determined by Bio-Rad protein assay kit (Bio-Rad Laboratories, Inc. Hercules, Calif.). The supernatant protein was aliquoted and stored at −70° C. until used. The protein was dialyzed routinely against HBSS before being used for migration assay analysis.

SDS-PAGE and N-Terminal Protein Sequencing Analyses

The prepared mEndo and NulI supernatant protein were analyzed by SDS-PAGE. Each 60-μg of supernatant protein was mixed with Laemmli sample buffer (Sigma, St. Louis, Miss.) and heated at 95° C. for 3 min. The denatured protein was loaded on a 4-12% linear gradient pre-casted gel (Bio-Rad). The gel was stained with Gelcode blue stain reagent (VWR Scientific Products, Willard, Okla.) to visualize the protein bands. For mEndo N-terminal protein sequencing analysis, the protein was analyzed on SDS-PAGE in a similar way except that the protein was transferred to PVDF membrane (Bio-Rad). The membrane was stained with 0.1% Coomassie blue R-250 in 40% methanol and 1% acetic acid for 2 min followed by 4 destaining washes with 50% methanol for 15 min. per wash. The membrane was air-dried and the 20 Kd protein band corresponding to murine endostatin was subjected to N-terminal protein sequencing analysis by automated Edman degradation using Perkin-Elmer Applied Biosystems (Protein Sequencing Midwest Analytical, Inc., St. Louis, Miss.).

ELISA Detection of mEndo Secretion

Secretion of murine endostatin was determined routinely by a murine endostatin ELISA kit named ACCUCYTE Murine Endostatin (Cytlmmune Sciences, Inc., College Park, Md.) according to the manufacturer's procedure. Following the washing of the plate, the analysis was carried out in triplicate on a 96-well ELISA plate with the unknown samples diluted by 4, 16, or 64-fold with final 50% of diluent 1 and 25% of diluent 2. 100-μl each of mEndo standard and diluted unknown sample were dispensed into a designated well. Following addition of biotin mEndo-conjugate and anti-mEndo antibody, the plates were incubated at room temperature for 3 hours. After the plate was washed, streptavidin-alkaline phosphates was added. The plate was incubated at room temperature for 40 min followed by 20-min color development in the streptavidin-alkaline phosphatase reaction mixture. The absorbance was determined at 492 nm by ELISA reader (Bio-Rad). The standard curve was established and the concentration of the unknown samples was determined by extrapolation from murine endostatin standard curve.

Migration Assay

Cell migration was assessed in 48-well chemotaxic chambers (Neuroprobe, Cabin John, MD) as described (Polverine, et al., Meth. Enzymol, Vol. 198, pgs. 440-441 (1991)). Polycarbonate (8μ) membrane (VWR Scientific Products) was coated with bovine collagen type I (Becton Dickinson Labware, Bedford, Mass.) in the following procedure. Polycarbonate membrane was soaked in 0.5 M acetic acid overnight. The membrane was rinsed with PBS. Collagen type I (bovine) was diluted in 0.2 N acetic acid to the final concentration of 0.1 mg/ml. The membrane was soaked in 0.1 mg/ml collagen for 2 min and air-dried. Low passage (passage 1 to 4) of HUVEC were cultured in M200 supplemented with LSGS (Cascade) until migration assay. Cells were suspended in migration assay medium, M199 plus 1% FBS to the cell density of 2×10⁵ cells/ml and pre-incubated in the presence or absence of tested supernatant proteins, mEndo or NulI in 37° C. 5% CO₂ incubator for 30 min. VEGF165 (R&D Systems, Minneapolis, Minn.) at various concentrations were prepared in migration assay medium and added into the bottom chamber. After assembly with collagen type I coated polycarbonate membrane in between the top and bottom chambers, 50 μl of pre-incubated cell suspension were added to the top chamber. The membrane was removed after 5 hours of incubation and was stained with Diff-Quik (VWR). The seeded cells on the top chamber were removed by wiping with tissue. Cells that migrated through the membrane to the bottom chamber were counted using BioQuan image system under the microscope. The basal migration was determined with migration assay medium in the absence of any tested substance, mEndo, NulI, or angiogenic factor, VEGF165.

Results and Discussion

Generation of a Recombinant Adenoviral Vector Encoding Murine Endostatin

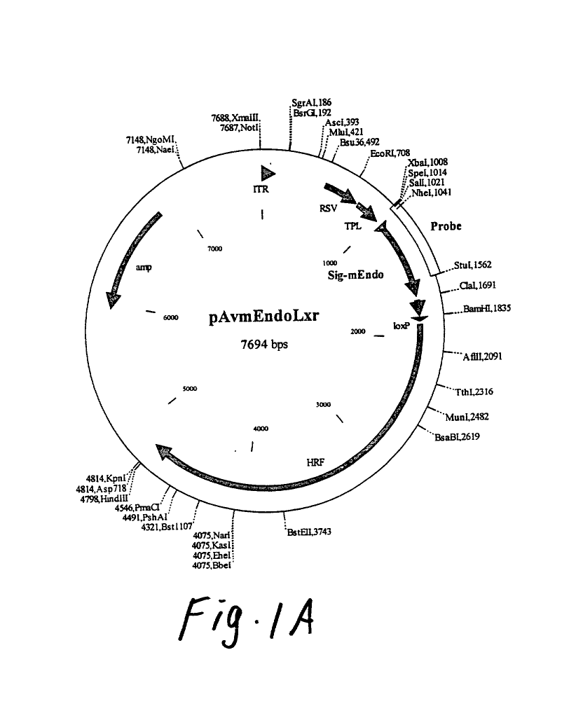

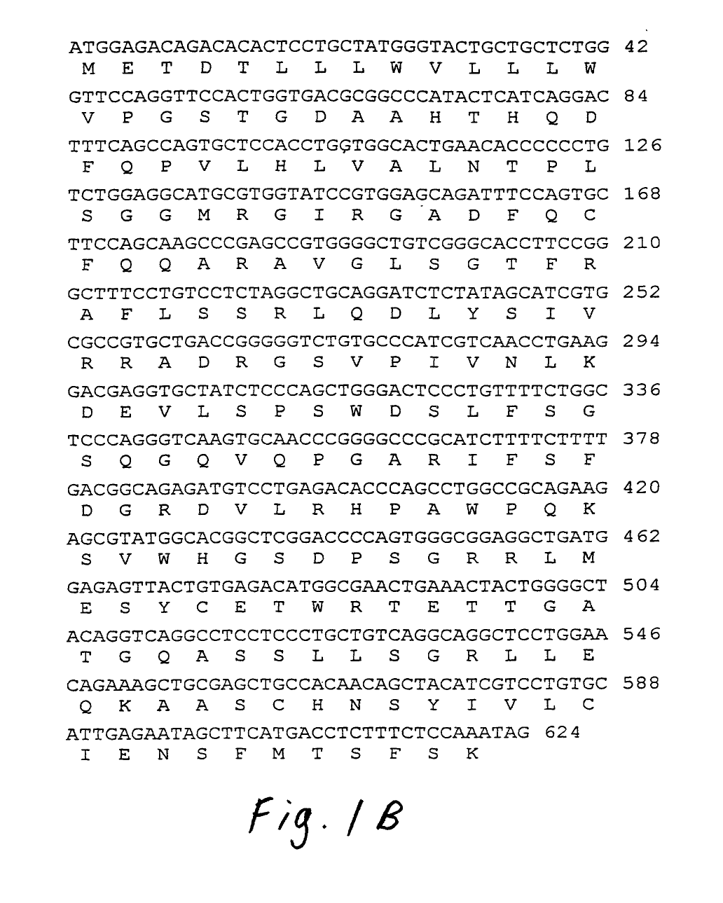

Murine endostatin cDNA was PCR generated from the C-terminus of mouse α1(XVIII) collagen clone ID #748987 from GenomeSystems. The cDNA was assembled with murine Ig-kappa leader to generate sig-mEndo chimeric for the secretion of murine endostatin protein by PCR splice overlap extension (Horton, et al., 1990). The sig-mEndo chimeric DNA was cloned into the Nhel and ClaI sites of the adenoviral shuttle plasmid, pAvF91xr to create pAvmEndoLxr (FIG. 1A). The entire sig-mEndo chimeric sequence was confirmed by auto sequencing analysis. The consensus sequence and derived protein sequence are shown in FIG. 1B. The adenoviral vector encoding sig-mEndo chimeric was generated by the “Quick Cre/Lox 2 plasmid system” in 293 cells by transient transfection with pcmvE2a, pCre, pSQ3 and pAv3mEndoLx through Cre/Lox mediated recombination. The generated vector was RCA negative. The correct genome structure of generated Av3mEndo vector was confirmed by restriction digests and Southern Blot analysis (FIGS. 3A and B).

Expression and Secretion of Murine Endostatin

Av3mEndo mediated murine endostatin expression was characterized by Northern Blot analysis in vector transduced A549 cells (FIG. 4). The expected sig-mEndo message was only identified in Av3mEndo but not in control, untreated or Av3NulI vector transduced cells. The expression and secretion of murine endostatin was characterized further in vector transduced S8 cells. As shown in FIG. 5, the supernatant proteins of Av3mEndo and Av3NulI vector transduced S8 cells were compared on SDS-PAGE. A distinct 20 Kd protein band corresponding to the correct size of murine endostatin was only identified in Av3mEndo but not in Av3NulI transduced cells. This 20 Kd protein was subjected to N-terminal protein sequencing analysis and demonstrated the cleavage of the signal peptide from murine endostatin at the expected site. The secretion of murine endostatin was also characterized in Av3mEndo transduced Hep3B cells and demonstrated the endostatin expression and secretion (FIG. 6).

Functional Characterization of Murine Endostatin in vitro

The biological activity of the mEndo supernatant protein was characterized in both HUVEC proliferation and migration assay. No inhibition was observed in bFGF induced HUVEC proliferation assay in the presence of mEndo supernatant protein (data not shown); however, mEndo supernatant protein demonstrated potent inhibition effect against VEGF165 induced HUVEC migration in comparison with no effect by NulI supernatant protein as shown in FIG. 7.

### Example 2

Materials and Methods

Adenoviral Vectors

Av3mEndo vector encoding secreted murine endostatin was constructed as described in Example 1. The control vector, Av3NulI possesses the same backbone gene except that no coding sequence was in the expression cassette. Vectors used in this report were bulked up by the tumor targeting group or Gene Therapy Core Technologies at GTI, which are listed below, Av3mEndo, Lot#TCA74A&B, 1.4×10¹¹ particles/ml, particle/pfu ratio of 33.6; Av3mEndo, Lot#CTC7-98, 2.18×10¹² particles/ml, particle/pfu ratio of 77.3; Av3NulI, Lot#TCA75, 2.4×10¹² particles/ml, particle/pfu ratio of 21.8; and Av3NulI, Lot#TCA29B, 3.64×10¹² particles/ml, particle/pfu ratio of 37.5.

ELISA Detection of mEndo Secretion

Secretion of murine endostatin was determined routinely by murine endostatin ELISA kit (CytImmune Sciences, Inc., College Park, Md.) according to the manufacturer's procedure. Following washing of the plate, the analysis was carried out in triplicate on a 96-well ELISA plate with the unknown samples diluted by 4, 16, or 64fold with final 50% of diluent 1 and 25% of diluent 2. Each 100-μl of mEndo standard and diluted unknown sample was dispensed into designated well. Following addition of biotin mEndo-conjugate and anti-mEndo antibody, the plates were incubated at room temperature for 3 hours. After the plate was washed, the streptavidin-alkaline phosphatase was added. The plate was incubated at room temperature for 30 min. followed by 20-min. color development in the streptavidin-alkaline phosphatase reaction mixture. The absorbance was determined at 492 nm by ELISA reader (Bio-Rad). The standard curve was established and the concentration of the unknown samples was determined by extrapolation from murine endostatin standard curve.

Colon Liver Metastasis Model

All the animal studies were designed by tumor targeting group at Genetic Therapy, Inc. Male athymic CD-1 nude mice between 4 and 5 weeks of age were purchased from Charles River Laboratories (Wilmington, Mass.). The mice were maintained in a HEPA filtered environment with cages, food and bedding sterilized by autoclaving. Animal diets were purchased from Harlan Teklad (Madison, Wis.). Ampicillin 5% (v/v) (Sigma, St. Louis, Mo.) was added to the autoclaved drinking water. Mice were treated with Av3mEndo (Lot#TCA74A&B) (n=20) or the control, Av3NulI (Lot#TCA75) vectors (n=20) at 2×10¹¹ particles/mouse by tail vein injection at final volume of 100 μl. The controls (12 mice) were also carried out with HBSS saline alone at 100 μl per mouse. Ten days post vector injection, the blood endostatin level was determined in all mice by murine endostatin ELISA kit (Cytlmmune, College Park Md.). Thirteen days post-vector injection, poorly differentiated human stage IV colon adenocarcinoma (T3N1 M1) was implanted by surgical orthotopic implantation to the top of the ascending colon approximately 1 cm away from the cecum after the serosa of the site has been stripped. The intestine was then returned to the abdomen and the abdomen was closed in one layer with sterile 6-0 surgical sutures. After tumor implantation, there were 11, 17, and 16 mice that survived in the HBSS, Av3NulI, and Av3mEndo treated groups, respectively. The animal survival was then monitored throughout the study. The % survival was calculated based on 100% survival of those mice survived after orthotopic surgical tumor implantation.

B16F10 Lung Metastasis Model

Male C57BL6/J mice at the age between 8 to 9 weeks old were purchased from Harlan Sprague Dawley, Inc. (Indianapolis, Ind.). Mice were treated with Av3mEndo (Lot#TCA74A&B), or the control, Av3NulI (Lot#TCA75) vectors at 2×10¹¹ particles/mouse by tail vein injection at final volume of 100 μl. The controls were also carried out with HBSS saline alone at 100 μl per mouse. Two days post vector injection, lung metastasis was established by tail vein injection of B16F10 cells suspension at 5×10⁴ cells/mouse. Fourteen days post tumor cell injection, all mice were sacrificed and autopsied. Liver samples were collected for liver transduction analysis. Blood was collected for murine endostatin determination. Lung was collected for surface metastases determination by stereomicroscope.

Liver Transduction Analysis

Liver transduction was performed by Southern blot analysis following the standard protocols by Genetic Therapy, Inc. Gene Therapy Core Technologies. Genomic DNA was isolated from frozen liver samples of control, Av3NulI and Av3mEndo treated groups using the Qiagen Genomic DNA Isolation Kit. (Qiagen, Inc., Valencia, Calif.) Frozen liver was minced and treated with protease at 55° C. for 18 hours in the Hybaid oven. After centrifugation, DNA was then isolated from the supernatant using a Qiagen column. DNA was digested with NcoI and resolved on 1% agrose-TAE. After transferred to a nylon membrane, the membrane was prehybridized in 5× Denhardts, 6× SSC, 10 mM EDTA, 0.5% SDS, and 0.1 mg/ml salmon sperm DNA at 68±2° C. for 2 hours. The membrane was then hybridized with a 554 bp [³²P]-labeled sig-mEndo internal probe at 68±2° C. and washed in SSC/SDS containing buffers at 68±2° C. following the standard protocol. The sig-mEndo Internal probe was prepared from pAvmEndoLxr digested by XbaI and StuI at 37° C. for 4 hours and gel purified. The standards of various numbers of copies of sig-mEndo chimeric DNA per hepatocyte were prepared with known amounts of pAvmEndoLx shuttle plasmid added to 10 μg hepatocyte genomic DNA for quantitative analysis. The ³²P radioactivity of sig-mEndo chimeric DNA band was determined by Phosphor Imager analysis. The copy number of sig-mEndo chimeric DNA per hepatocyte was extrapolated from the standard curve generated as described above.

B16F10 Subcutaneous Model

Male C57BL6/J mice at the age between 11 to 12 weeks old were purchased from Harlan Sprague Dawley, Inc. (Indianapolis, Ind.). Mice were treated with Av3mEndo (Lot#CTC7-98), or the control, Av3NulI (Lot#TCA29B) vectors at 6×10¹⁰ particles/mouse by tail vein injection at final volume of 100 μl. The controls were also carried out with HBSS saline alone at 100 μl per mouse. Two days post vector injection, B16F10 cells were implanted sucutaneously at 5×10⁵ cells per mouse. Tumor volume was measured throughout the study. Liver samples were collected for liver transduction analysis. Blood was collected for murine endostatin determination.

Results and Discussion

Naturally Occurring Colon Liver Metastasis Model

To determine if systemic delivery of Av3mEndo vector can produce systemic secretion of murine endostatin above the endogenous level, male athymic CD-1 nude mice were treated with Av3mEndo vector at 2×10¹¹ particles/mouse by tail vein injection. The controls were carried out with Av3NulI vector at identical dose or with HBSS at equal volume. Blood level of murine endostatin was determined by ELISA on day 10 post vector injection. As shown in FIG. 8A, mice treated with Av3mEndo vector demonstrated 5 to 8-fold higher levels of murine endostatin than the controls, Av3NulI or HBSS treated mice.

Because the liver is probably the site with the highest and longest adenoviral vector transduction in body, a higher local secretion of endostatin in liver than other sites was expected. The systemic delivery Av3mEndo might bring more benefits toward liver metastasis reduction than metastasis in other sites. Therefore, we tested the strategy of systemic delivery of Av3mEndo in this naturally occurring metastasis model. Poorly differentiated human stage IV colon adenocarcinoma (T3N1M1) was implanted by surgical orthotopic implantation to the top of the ascending colon of vector treated mice on day 13 post vector injection. Tumor implanted mice usually develop micro liver metastasis 7-10 days post orthotopic transplantation with a few cases with lymph node metastasis. Liver metastasis involves all liver lobes and metastatic lesions overcome all liver tissues, and liver volume increases 3 to 10 times. Mice normally died of serious late stage liver metastasis. As shown in FIG. 8B, mice treated with Av3mEndo vector before tumor implantation demonstrated higher survival rates than the controls of Av3NulI and HBSS treated mice. Fifty-three days post tumor implantation, 62.5% of the mice survived in the Av3mEndo treated group, while only 11.8% and 18.1% of the mice survived in Av3NulI and HBSS treated groups, respectively. 25% of the mice of the Av3mEndo treated group survived for 188 days after tumor implantation. These mice then were sacrificed, and then were examined for primary tumors at the original site of implantation at the top of the ascending colon of the colon adenocarcinoma, and were examined for liver metastases. In each of the mice that survived 188 days after tumor implantation, no primary tumors in the colon were found, and no liver metastases were found.

The correlation between endostatin levels expressed by the mice, and survival of the mice was plotted in FIG. 8C. As shown in FIG. 8C, mice which survived for longer periods of time, including those which survived 188 days after implantation of the colon adenocarcinoma, in general had blood endostatin levels of 200 ng/ml or more 10 days post vector administration. The circulating level of endostatin (140 ng/ml±64 ng/ml) was detected in surviving mice 144 days after implantation of the colon adenocarcinoma. The results clearly demonstrated that secreted endostatin by Av3mEndo treatment provided prolonged survival in this naturally occurring liver metastasis model.

B16F10 Melanoma Lung Metastasis Model

The B16F10 lung metastasis model was established in C57B16/J mice by tail vein injection of 5×10⁴ cells per mouse. Two days before tumor implantation, the mice were treated with Av3mEndo vector by tail vein injection at 2×10¹¹ particles per mouse. Controls were carried out with either equal volume of HBSS or the equal amount of Av3NulI vector. Fourteen days post tumor implantation, blood was collected from all animals and analyzed by mEndo ELISA. As shown in FIG. 9A, the mice treated with Av3mEndo vectors all demonstrated higher level of murine endostatin at an average of 708±435 ng/ml. In contrast, the control mice treated with HBSS and Av3NulI only showed the endogenous level of murine endostatin at an average of 50±15 and 56±19 ng/ml, respectively.

Fourteen days post tumor implantation, all mice were sacrificed and autopsied. Liver transduction was determined from Av3NulI and Av3mEndo treated mice by mEndo Southern Analysis. All Av3mEndo treated mice demonstrated Av3mEndo liver transduction with the average of 11±4 copy number of mEndo DNA per hepatocyte with respect to 0 copy of mEndo DNA per hepatocyte in the Av3NulI treated mice.

Lung surface metastasis was determined in all mice. As shown in FIG. 9B, the control mice treated with HBSS showed the highest number of lung metastases at an average of 109±65 number of lung metastases/mouse. Mice treated with Av3mEndo vectors demonstrated the reduction of lung metastases to the average of 36±18 number of lung metastases/mouse (33% relative to HBSS control). However, mice treated with Av3NulI vectors also demonstrated the reduction of lung metastases to the average of 42±28 number of lung metastases/mouse (39% relative HBSS control). This indicated that the majority of anti-metastasis effect (61%) was caused by Av3 backbone vector and some reduction (6%) was caused by murine endostatin secretion. The current study does prove that Av3mEndo expressed and secreted functional murine endostatin was demonstrated in vitro. Systemic administration of Av3mEndo demonstrated sustained blood level of endostatin as demonstrated on day 16 post vector injection. The results support that anti-angiogenesis gene therapy of angiogenic inhibitor gene delivery may provide a means to reduce lung metastasis.

Liver transduction also was correlated to levels of blood endostatin in the Av3mEndo treated mice. As shown in FIG. 9C, mice which demonstrated increased liver transduction, as indicated by copy number per hepatocyte, had higher levels of endostatin in the blood.

B16F10 Melanoma Subcutaneous Model

The B16F10 subcutaneous model is one the models which demonstrated potent inhibition against tumor growth by systemic delivery of endostatin protein subcutaneously (O'Reilly, et al. (1997). If Av3mEndo vector can provide systemic secretion of endostatin at the therapeutic level, the antitumor effect by systemic delivery of Av3mEndo vector should be reproducible. C57BLJ/6 mice were treated with Av3mEndo, or control Av3NulI vectors at 6×10¹⁰ particles/mouse, or equal volume of HBSS by tail vein injection. Two days post vector injection, subcutaneous tumor cells were implanted at 5×10⁴ cells per mouse. Tumor volume was measured and recorded twice a week throughout the study. As shown in FIG. 10A, Av3mEndo treated mice demonstrated the smallest tumor volume in average among the three treated groups. At the end of the study, the blood level of murine endostatin was determined. As shown in FIG. 10B, all Av3mEndo treated mice demonstrated higher blood levels of murine endostatin than the control mice. However, the level was not much above the level in the control mice except one mouse with a blood level of endostatin of 500 ng/ml. The mouse with endostatin at 500 ng/ml showed the smallest tumor volume among all treated mice. All Av3mEndo treated mice demonstrated liver transduction by Av3mEndo at an average of 1.2±0.7 copies per hepatocyte at the end of study.

### Example 3

Ten male athymic CD-1 nude mice between 8 and 10 weeks of age (Charles River Laboratories, Wilminton, Mass.) were injected with Av3mEndo vector at 2×10¹¹ particles per mouse by tail vein injection. The mice were divided into two groups, Group A and Group B. Blood was collected by retro-orbital bleeding from Group A at 0, 0.5, 1.5, 2.5, 4, 6, 8, 10, and 12 weeks and from Group B at 1, 2, 3, 4, 5, 7,9, and 11 weeks. An endogenous level of blood endostatin was established by a control mouse without the Av3mEndo treatment. Blood murine endostatin was determined by ELISA as described in Example 2.

As shown in FIG. 11, the peak level of murine endostatin secretion was at day 3, post-vector injection. The steady-state level of endostatin was above 300 ng/ml and decreased gradually to the level of 150 ng/ml over 80 days post-injection. In contrast, the control mouse without Av3mEndo treatment showed only the endogenous level of endostatin which is below 50 ng/ml. The results demonstrated that a single administration of the Av3mEndo vector by tail vein injection could provide a sustained increased blood level of endostatin for a period of over 80 days.

### Example 4

Materials and Methods

PCR and Assembly of Human Endostatin cDNA and BM40 Basement Protein Leader Sequence

The human endostatin cDNA was PCR amplified from the cDNA of human 60 1 (XVIII) collagen. The human liver cDNA was generated from human liver poly A RNA (Clonetech, Palo Alto, Calif.) by reverse transcriptase polymerase chain reaction (RT-PCR). The reverse transcription was carried out with the primer of 5′-TTT TTT TTT CAG TGT AAA AGG TC-3′using the Perkin Elmer RT-PCR kit (Perkin Elmer Applied Biosystems, Foster City, Calif.) for 1 cycle in the following conditions: room temperature for 10 min, 42° C. reverse transcribing for 3 min, 99° C. denaturation for 5 min, 5° C. cooling for 5 min, and hold at 4° C. until the cDNA was ethanol precipitated and resuspended. The 790 bp human endostatin cDNA fragment was PCR amplified from the prepared cDNA with the primers of 5′-CAG ATG ACA TCC TGG CCA G-3′ and 5′ CTA TAC AGG AAA GTA TGG CAG C-3′. PCR was carried out for 35 cycles in the following condition: 95° C. hot start for 3 min, 80° C. for 3 min followed by the addition of Pfu DNA polymerase (Stratagene, La Jolla, Calif.), 95° C. denaturation for 1 min, 55° C. annealing for 1 min, and 72° C. extension for 3 min. The 790 bp human endostatin cDNA fragment was gel purified and reamplified as described except using the annealing temperature of 58° C. The 790 bp human endostatin cDNA fragment was gel purified and cloned into PCR-Script Amp SK⁺ using PCR-Script Cloning Kits (Stratagene) according to the manufacturer's procedure to generate pcrhend 1. The human endostatin cDNA region of the pcrhend 1 plasmid was confirmed with the direct sequencing analysis by Gene Therapy Core Technologies Molecular Core Laboratory at Genetic Therapy, Inc. Gaithersburg, Md.

The human endostatin cDNA fragment was assembled with human BM40 basement protein leader according to the following procedure. The BM40 basement protein leader was generated by annealing 2 pieces of synthesized oligonucleotides, 5′GCC AAG CTT CCA TGA GGG CCT GGA TCT TCT TTC TCC TTT GCC TGG CCG GGA GGG CTC TGG CAG CCC CTC AGC AAG AAG CGC TCG CTC ACA GCC ACC GCG ACT TCC AGC CGG TGC TCC A-3′ (sense), and 5′-CCA GGT GGA GCA CCG GCT GGA AGT CGC GGT GGC TGT GAG CGA GCG CTT CTT GCT GAG GGG CTG CCA GAG CCC TCC CGG CCA GGC AAA GGA GAA AGA AGA TCC AGG CCC TCA TGG AAG CTT GGC-3′ (antisense) followed by Hind III and Sex A1digestion. Th digested BM40 basement protein leader was cloned into Hind III and Sex A1 sites of pcrhend 1 to generate pBmpcrhen plasmid. The entire sig-hEndo region of the pBmpcrhen plasmid was confirmed with the direct sequencing analysis by Gene Therapy Core Technologies Molecular Core Laboratory at Genetic Therapy, Inc. The adenovial shuttle plasmid pAV1bmhend1x was generated by substitution of the Factor IX (F9) containing sequence with the sig-Endo containing sequence in pAvF9Lxr adenoviral shuttle plasmid in the following procedure. An 800 bp fragment containing sig-hEndo sequence was generated from pBmpcrhen by SacI digestion followed by Klenow fill in and Sal I digestion. The pAvF9Lxr plasmid was digested with Bam HI restriction enzyme followed by Klenow fill in and digested with Sal I restriction enzyme to remove F9 containing sequences. The two digested fragments were gel purified and ligated to generate pAV1bmhend1x. (FIG. 12A).

The recombinant Av3bmhend1x (with E1, E2a, and E3-deletions) encoding the sig-hEndo chimeric protein was generated by the “Quick Cre/Lox two plasmid system” according to the following procedure. The plasmids pAV1bmhend1x and pSQ3 were linearized first with Not I and Cla I restriction enzymes, respectively. The S8 cells were pretreated with 0.3 μM dexamethasone 24 hours before the transient transfection that was performed on the 6-well plate at 4×10⁵ S8 cells per well using LipofectAMINE PLUS Reagent (Life Technologies, Rockville, Md.). The lipofectamine complexed DNA was prepared with 1 μg of linearized pSQ3, 0.5 μg pCre, and 0.5 μg linearized pAV1bmhend1x, and 6 μl of lipofectamine according to the manfacturer's procedure (Life Technologies). The S8 cells were incubated with lipofectamine complexed DNA at 37° C. for 4.5 hours. The lipofectamine complexed DNA was removed and the cells were washed with PBS. The transfected S8 cells were cultured at 37° C. with 5% CO₂ until the CPE was observed. The cells and the medium were harvested by scraping. The crude viral lysate was prepared by five cycles of freezing and thawing. The Av3bmhend1x was re-amplified in S8 cells with 0.3 μM dexamethasone in Richcter's CM medium containing 5% FBS until CPE was observed.

Preparation of the Purified Av3bmhend1x

The purified Av3bmhend1x vector was scaled up by the tumor targeting group at Genetic Therapy, Inc. The adenoviral titer (particles/ml) and biological titer (pfu/ml) were determined as described (Mittereder, et al., 1996) by Genetic Therapy, Inc.

Preparation of hEndo Supernatant Protein from Vector Transduced Cells

The hEndo supernatant protein was prepared from Av3bmhend1x transduced S8 cells in the same procedure as described in Example 1.

SDA-PAGE and N-Terminal Protein Sequencing Analyses

The prepared hEndo supernatant protein was analyzed by SDS-PAGE and N-terminal protein sequencing analyses as described in Example 1.

Results

Generation of a Recombinant Adenoviral Vector Encoding Human Endostatin

Human endostatin cDNA was RT-PCR generated from the C-terminus of cDNA of human α1 (XVIII) collagen from human liver poly A RNA. The human BM40 basement protein leader was generated from two pieces of synthesized oligonucleotides. The annealed human BM40 basement protein leader was cloned 5′ of the human endostatin cDNA to generate sig-hEndo chimeric protein for the secretion of human endostatin protein. The sig-hEndo chimeric DNA was cloned into the adenoviral shuttle plasmid, pAvF91xr to create pAV1bmhend1x (FIG. 12A). The entire sig-hEndo chimeric sequence was confirmed by auto sequencing analysis. The consensus sequence and derived protein sequence are shown in FIG. 12B. The adenoviral vector encoding sig-hEndo chimeric was generated by the “Quick Cre/Lox 2 plasmid system” in S8 cells by transient transfection with pCre, pSQ3, and pAV1bmhend1x through Cre/Lox mediated recombination.

Expression and Secretion of Human Endostatin

Av3bmhend1x mediated human endostatin expression and secretion was characterized in vector transduced S8 cells. As shown in FIG. 13, the supernatant protein of Av3bmhend1x, i.e., human endostatin, was analyzed by SDS-PAGE (lanes 1-8 and 10). Each 20 μg of supernatant protein was analyzed on 4 to 12% linear gradient precasted gel. The protein standard marker was run on lane 9. The SDS-PAGE was transferred to a polyvinylidene fluoride membrane. The membrane was stained with Coomassie blue R-250. The 20 kDa protein bands, corresponding to the correct size of human endostatin, were excised from a membrane blot and subjected to N-terminal protein sequencing analysis. The determined protein sequence is shown at the bottom of FIG. 13 with arrows marked at the beginning of the N-termini of three major secreted proteins, 50% containing the additional amino acid residues APQQEALA, 25% containing residues LA, and 25% containing no residues from human BM40 basement protein signal peptide. The 20 kDa protein was not shown in the supernatant protein from Av3NulI cells. (FIG. 5). The results demonstrated that S8 cells transduced with Av3bmhend1x expressed and secreted human endostatin after it was processed from human BM40 basement protein signal peptide.

The disclosure of all patents, publications, (including published patent applications), and database accession numbers and depository accession numbers referenced in this specification are specifically incorporated herein by reference in their entirety to the same extent as if each such individual patent, publication, and database accession number, and depository accession number were specifically and individually indicated to be incorporated by reference.

It is understood, however, that the scope of the present invention is not to be limited to the specific embodiments described above. The invention may be practiced other than as particularly described and still be within the scope of the accompanying claims.

What is claimed is:

1. An adenoviral vector including a DNA sequence encoding endostatin.

2. The vector of claim 1 and further comprising a DNA sequence encoding a secretion signal peptide immediately 5′ to said DNA sequence encoding endostatin.

3. The vector of claim 2 wherein said secretion signal peptide is the secretion signal peptide of Ig-Kappa.

4. A method of providing for expression of endostatin in a host, comprising:
administering to a host the adenoviral vector of claim 1.

5. The method of claim 4 wherein said adenoviral vector is administered systemically.

6. The method of claim 4 wherein said adenoviral vector is administered regionally.

7. The method of claim 4 wherein the host is a mammalian host.

8. The method of claim 7 wherein said adenoviral vector is administered in an amount effective to provide for expression of endostatin in an amount of up to 1,000,000 ng/ml.

9. The method of claim 7 wherein said adenoviral vector is administered in an amount effective to provide for expression of said endostatin in said mammalian host in an amount of at least about 200 ng/ml.

10. The method of claim 9 wherein said endostatin is expressed in said mammalian host in an amount of from about 200 ng/ml to about 500 ng/ml.

11. A method of treating a tumor in a host, comprising:
administering to said host the adenoviral vector of claim 1.

12. The method of claim 11 wherein said adenoviral vector is administered systemically.

13. The method of claim 11 wherein said adenoviral vector is administered regionally.

14. A method of treating tumor metastases, comprising:
administering to a host the adenoviral vector of claim 1.

15. The method of claim 14 wherein said adenoviral vector is administered systemically.

16. The method of claim 14 wherein said adenoviral vector is administered regionally.

17. The method of claim 14 wherein said tumor metastasis is a tumor metastasis found in the liver.

18. The method of claim 14 wherein said adenoviral vector is administered in an amount effecctive to provide for expression of endostatin in said host at a level of up to 1,000,000 ng/ml.

19. The method of claim 14 wherein said adenoviral vector is administered in an amount effective provide for expression of endostatin in said host at a level of at least about 200 ng/ml.

20. The method of claim 19 wherein said endostatin is expressed in said host at a level of from about 200 ng/ml to about 500 ng/ml.

21. A method of treating colon cancer metastases in a host, comprising:
administering to said host the adenoviral vector of claim 1.

22. The method of claim 21 wherein said adenoviral vector is administered systemically.

23. The method of claim 21 wherein said adenoviral vector is administered regionally.

24. The method of claim 21 wherein said colon cancer metastasis is a colon cancer metastasis found in the liver.

25. The method of claim 21 wherein said adenoviral vector is administered in an amount effective to provide for expression of endostatin in said host at a level of up to 1,000,000 ng/ml.

26. The method of claim 21 wherein said adenoviral vector is administered in an amount effective to provide for expression of said endostatin in said host in an amount of at least about 200 ng/ml.

27. The method of claim 26 wherein said endostatin is expressed in said host in an amount of from about 200 ng/ml to about 500 ng/ml.

28. A method of expressing endostatin in a cell, comprising:
administering to a cell the adenoviral vector of claim 1.

29. The method of claim 28 wherein said cell is a mammalian cell.

30. The method of claim 29 wherein said cell is an A549 cell.

31. The method of claim 29 wherein said cell is a Hep3B cell.
